(19) United States
(12) Patent Application Publication
(10) Pub. No.: US 2006/0241733 A1
(43) Pub. Date: Oct. 26, 2006

(54) Atrial pacing lead

(75) Inventors: Yongxing Zhang, Maple Grove, MN (US); Yunlong Zhang, Mounds View, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/113,715

(22) Filed: Apr. 25, 2005

(51) Int. Cl. A61N 1/05

(52) U.S. Cl. 607/122

(57) ABSTRACT

A lead includes a lead body having an expandable section. A plurality of electrodes are disposed on the expandable section. The expandable section is adapted to expand against an inner surface of a heart so as to position at least one of the plurality of electrodes at or near an SA node of the heart.

### FIELD OF THE INVENTION

This invention relates to the field of medical leads, and more specifically to an atrial lead.

### BACKGROUND

Leads implanted in or about the heart have been used to reverse certain life threatening arrhythmia, or to stimulate contraction of the heart. Electrical energy is applied to the heart via electrodes on the leads to return the heart to normal rhythm.

For example, atrial pacing is accomplished by locating an electrode in the right atrium. However, there are limitations to present techniques. For example, the pacing stimuli may not be in line with the right atrium (RA) conduction path and the applied stimula cannot reach the left atrium (LA). This prevents efficient, synchronized RA-LA activation.

### SUMMARY

A lead includes a lead body having an expandable section and a plurality of electrodes disposed on the expandable section. The expandable section is adapted to expand against an inner surface of a heart so as to position at least one of the plurality of electrodes at or near an SA node of the heart.

### BRIEF DESCRIPTION OF THE DRAWINGS

FIG. 1 shows a partial cross-section view of a heart.

FIG. 2 shows a view of a lead, according to at least one embodiment, implanted within a heart.

FIG. 3 shows a side view of a portion of the lead of FIG. 2.

FIG. 4 shows a side view of a portion of the lead of FIG. 2.

FIG. 5 shows a side view of a portion of a lead according to at least one embodiment.

FIG. 6 shows a side view of the lead of FIG. 5.

FIG. 7 shows a cross-section view of a lead according to at least one embodiment.

FIG. 8 shows a cross-section view of a lead according to at least one embodiment.

FIG. 9 shows a side view of a portion of a lead according to at least one embodiment.

FIG. 10 shows a side view of the lead of FIG. 9.

FIG. 11 shows a side view of a lead according to at least one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

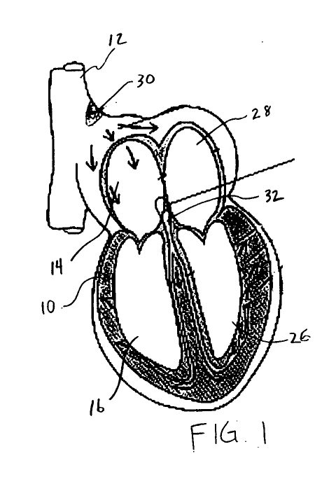

FIG. 1 shows a cross-sectional view of a heart 10. Heart 10 includes a superior vena cava 12 (SVC), a right atrium 14 (RA), a right ventricle 16, a left ventricle 26, a left atrium 28, and a sin θ-atrial (SA) node 30. The SA node 30 is located in the posterior wall of the right atrium 14 near the junction of the superior vena cava 12 and the right atrium 14. The superior vena cava 12 has a cross-sectional oval shape at the junction with the right atrium 14. At the junction, the end-diastolic cross-sectional long axis at the SVC 12 ranges from about 16 mm to about 24 mm and its short axis varies from about 10 mm to about 23 mm, according to a 3D echocardiographic study. The long and short axes can change 10-15% from end diastole to end systole. The SA node 30 typically has a size of about 3 mm×4 mm×25 mm. The SA node 30 includes specialized cells that undergo spontaneous generation of action potentials at a rate of 100-110 action potentials (“beats”) per minute. The normal range for sinus rhythm is 60-100 beats/minute. Sinus rates below this range are termed sinus bradycardia and sinus rates above this range are termed sinus tachycardia.

The sinus rhythm normally controls both atrial and ventricular rhythm. Action potentials generated by the SA node spread throughout the left atrium and the right atrium, depolarizing this tissue and causing atrial contraction. The impulse then travels into the ventricles via the atrioventricular node 32. Specialized conduction pathways within the ventricle rapidly conduct the wave of depolarization throughout the ventricles to elicit ventricular contraction. Therefore, normal cardiac rhythm is controlled by the pacemaker activity of the SA node 30. Abnormal cardiac rhythms can occur when the SA node fails to function normally or when normal conduction pathways are not followed.

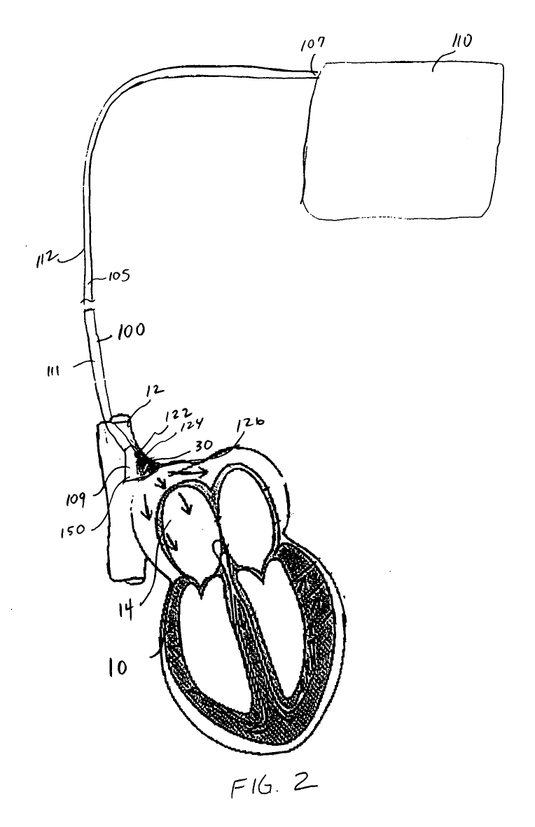

FIG. 2 shows a view of a lead 100, according to at least one embodiment, implanted within heart 10. In one embodiment, lead 100 is adapted to deliver pacing pulses to heart 10 via one or more electrodes 122, 124, 126. Lead 100 is part of an implantable system including a pulse generator 110, such as a pacemaker or defibrillator.

Pulse generator 110 can be implanted in a surgically-formed pocket in a patient's chest or other desired location. Pulse generator 110 generally includes electronic components to perform signal analysis and processing, and control. Pulse generator 110 can include a power supply such as a battery, a capacitor, and other components housed in a case. The device can include microprocessors to provide processing, evaluation, and to determine and deliver electrical shocks and pulses of different energy levels and timing for defibrillation, cardioversion, and pacing to heart 10 in response to cardiac arrhythmia including fibrillation, tachycardia, and bradycardia.

In one embodiment, lead 100 includes a lead body 105 extending from a proximal end 107 to a distal portion 109 and having an intermediate portion 111. Lead 100 includes one or more conductors, such as coiled conductors, to conduct energy from pulse generator 110 to heart 10, and also to receive signals from the heart. The lead further includes outer insulation 112 to insulate the conductor. The conductors are coupled to one or more electrodes 122, 124, 126. Lead terminal pins are attached to pulse generator 110. The system can include a unipolar system with the case acting as an electrode or a bipolar system.

In one embodiment, lead 100 includes an expandable member 150 disposed on the distal portion 109 of the lead body. As will be further explained below, expandable member 150 is adapted to secure at least one of electrodes 122, 124, 126 at or near the SA node 30 when the expandable member is secured at the location of the SA node at the junction of the superior vena cava 12 and the right atrium 14. The expandable section 150 expands such that at least some portions of the outer surface of the expandable section contact the inner surface of the heart at the SVC/RA junction to hold and secure the lead in place. Further, the expandable structure biases at least one of electrodes 122, 124, 126 against the heart surface to provide good contact with the SA node or its conduction fibers.

In one embodiment, electrodes 122, 124, 126 can include pacing electrodes adapted for delivering pacing pulses to the SA node 30. For instance, lead 100 can be designed for placement of pacing electrode 122 near or directly over the SA node to deliver energy pulses which provide optimal RA pacing. In some examples, the pulses provide synchronized bi-atrial activation. By pacing directly at the SA node, the present system can eliminate uncertainties regarding interatrial conduction time.

In some embodiments, lead 100 can be configured to allow both a stylet or catheter delivery. For example, an opening can be left through the middle of the lead to allow a stylet to be used.

In one embodiment, expandable member 150 can include a balloon or other structure that is expandable in vivo after the lead is properly inserted into the heart. In one embodiment, expandable member 150 can include a biocompatible material. In some embodiments, expandable member 150 can include a self-expanding structure made from a shape memory material, such as NiTi, for example.

The lead is designed such that after the lead is inserted and positioned at the junction of the SVC 12 and the RA 14, expandable member 150 is expanded. Expandable member 150, in its expanded state, has an outer dimension and shape that is designed such that the outer surface of the expandable member contacts the wall surfaces at the SVC 12/RA 14 junction so as to retain the lead and electrode 122 as implanted. Electrodes 122, 124, 126 are positioned relative to expandable member 150 such that at least one of the electrodes is proximate or directly over the SA node. Therapy can then be delivered directly to the SA node or the SA node conduction fibers via the electrode. In some embodiments, each electrode 122, 124, 126 can be independently coupled to the pulse generator and can be used to map the heart proximate the SA node and then one or more electrodes, located optimally, can be selectively chosen for SA node pacing.

In some embodiments, any of electrodes 122, 124, 126 can be used for sensing cardiac activity near the SA node. This information is delivered to the pulse generator and the pulse generator can use the information to deliver therapy pulses to the heart.

FIG. 3 shows a side view of lead 100 in accordance with one embodiment. In this view, expandable member 150 is in an unexpanded state and has a cross-sectional diameter approximately equal to the diameter of the lead 100. In this example, expandable section 150 can include a stent-like structure 302 mounted over a balloon 304 coupled to lead 100.

FIG. 4 shows a side view of lead 100 with stent-like structure 302 expanded. Balloon 304 can include an expanded shape that expands stent-like structure 302 from a narrow end which is coupled to the distal end of the lead to an expanded end at a distal end of the expandable member. The shape is designed to force at least one of electrodes 122, 124, 126, 128, 130, 132 against a wall of the heart proximate the SA node. The electrodes 122, 124, 126, 128, 130, and 132 can be independently coupled via conductors to the pulse generator. This allows the physician to use the electrodes to map the heart and the optimal electrode or electrodes can be chosen to deliver energy pulses to the SA node or the SA node conductive fibers. In one example, the shape defined by balloon 304 and structure 302 can be a bell-shape.

In one embodiment, the stent-like structure 302 can be etched from a single piece of metal starting material. In other embodiments, the stent-like structure is laser cut. In one embodiment, a flat starting material is first etched or laser cut and subsequently formed into a substantially tubular member. In one embodiment, a substantially flat starting material is welded into a substantially tubular member.

Possible starting material metals include, but are not limited to NITINOL, stainless steel, MP35N, tantalum, titanium, and alloy combinations of the above, etc. Materials other than metal, such as polymers, may also be used as starting materials. In one embodiment, surfaces that will be exposed inside the patient further include a coating of a bio-compatible material. Examples of bio-compatible materials include, but are not limited to, iridium oxide (IROX), platinum, titanium, tantalum, silver, etc. Portions of the stent-like structure can be insulated and electrodes can be mounted to the stent-like structure.

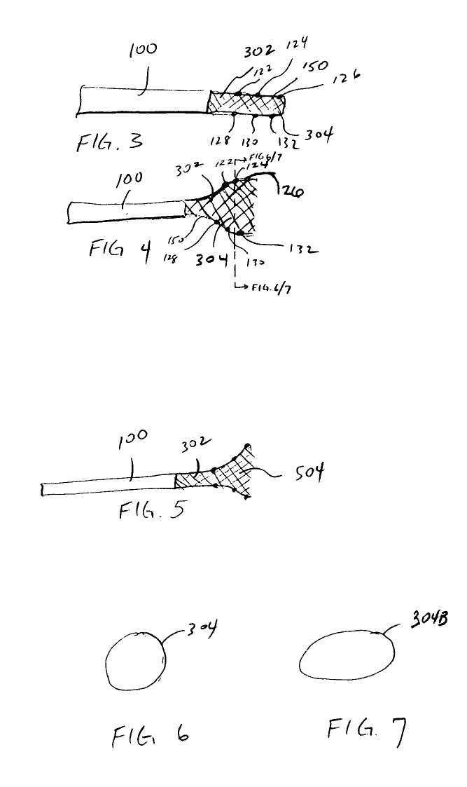

FIG. 5 shows another example of lead 100 according to one embodiment. In this example, a balloon 504 and stent-like structure 302 define a funnel shape.

FIGS. 6 and 7 show cross sections of balloon 304 and a balloon 304B, respectively, according to one or more embodiments. These cross sections are taken across lines 6/7 of FIG. 4. In any embodiments herein, a balloon can have these cross-section shapes or combinations of these shapes depending on the geometry of the SVC 12/RA 14 junction of the patient. As noted above, the SVC/RA junction has a cross sectional oval shape having a long axis and a short axis during a cardiac cycle. Accordingly, the shape of the expandable member 150 can be dimensioned so as to abut the inner surface of the heart walls at the junction to hold the lead and electrodes in place. As discussed above, one method of placing the lead is to map the evoked stimuli from the electrodes of the lead and utilize the electrode or electrodes that are optimally placed for SA node pacing. In any of the embodiments discussed herein, the lead can include 2, 4, 8, or more electrodes to help locate the optimal SA node pacing site.

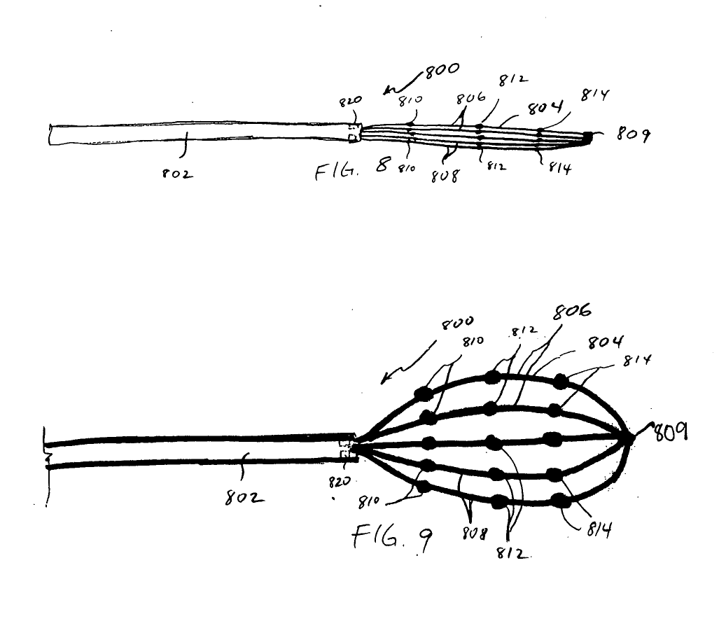

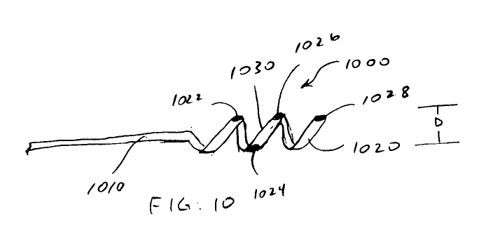

FIGS. 8 and 9 show side views of a lead 800 in an unexpanded state and an expanded state, respectively, in accordance with one embodiment. Lead 800 includes a lead body 802 and a plurality of splines 806, 808 that are coupled at their distal ends 809. The splines define an expandable section or expandable basket 804 having a plurality of electrodes 810, 812, and 814 exposed on an outer surface.

FIGS. 8 and 9 show splines 806, 808 at the distal end of lead 800. In one embodiment, the splines can be opened by manipulating an actuating suture extending through the lead body to expand the splines into the second, expanded orientation (FIG. 9) where the outer diameter of the basket structure has a greater diameter than the diameter of the distal end of the lead. This expanded orientation holds the lead in position at the junction of the SVC and the RA proximate the SA node. In some examples, splines 806, 808 can be shape memory material or biased members so as to be self-expanding. In one embodiment, a balloon can be located within the splines and inflated to expand the splines into position. In one embodiment, a seal 820 is located at the distal end of the lead 800 to seal the internal lumen of the lead. For example, the seal can include a valve proximal to the distal expandable portion within the lumen to prevent blood flow into the proximal end or a valve located near the distal end of the lumen.

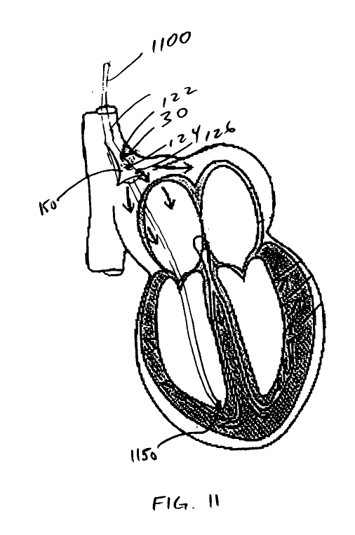

FIG. 10 shows a side view of a lead 1000, in accordance with one embodiment. Lead 1000 includes a lead body 1010 having a distal end 1020. Distal end 1020 includes an expandable or preformed section 1030. Preformed section 1030 includes a shape adapted to secure one or more electrodes 1022, 1024, 1026, 1028 proximate a junction of a superior vena cava and a right atrium. In some embodiments, preformed section 1030 includes a spiral shape with a substantially constant diameter D. In other embodiments, the preformed section can haven a spiral shape having an outer diameter which is narrower at its proximal end and wider at the distal end, or the spiral shape can go from wider to narrower.

Lead 1000 can include any features of the leads discussed above or below and the discussions are incorporated herein by reference. To preform section 1030 of lead 1000, the lead can be manufactured such that it is biased with the shape 1030. Thus, the lead naturally reverts to the pre-biased shape when it is implanted. For example, the lead body can be formed in the pre-biased shape or the conductor coils can be formed in the pre-biased shape to bias the lead body into the shape. A stylet or catheter can be used to implant the lead until the preformed shape is at the junction of the SVC and the right atrium. When the stylet or catheter is removed, the pre-formed shape 1030 returns to its pre-biased shape helping retain the lead in the implanted position, since in its expanded or biased orientation the shape defines an overall outer dimension greater than the dimension of the diameter of the distal end of the lead. Again, the electrodes can be used to map the heart and one or more electrodes can be chosen to deliver pacing to the SA node, such as discussed above.

In some embodiments, any of the leads discussed above can be used for mapping and locating a location for SA node pacing. Then a separate pacing lead can be introduced and actively fixated at the location identified by the mapping lead.

FIG. 11 shows a lead 1100 in accordance with one embodiment. Lead 1100 can include any features discussed above. Lead 1100 further includes a right ventricle electrode 1150. Electrode 1150 can be a pacing electrode or a defibrillation electrode.

The present system allows for mapping and for direct SA node pacing. In use, a lead, such as any lead discussed above, is implanted near the SA node and an expandable member on the lead is deployed at a junction between a superior vena cava and a right atrium. This causes one or more of a plurality of electrodes on the expandable section of the lead to be biased towards the SA node or a conduction path of the SA node. The electrodes can be independently coupled to a pulse generator to allow for mapping of the heart. Then one or more of the electrodes can be chosen to deliver pacing pulses directly to or proximate to the SA node.

The present lead allows for bi-atrial synchronized pacing utilizing a single electrode and the position of the electrode is optimized at the SA node due to the plurality of electrodes and the mapping function.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A lead comprising:
a lead body having an expandable section; and
a plurality of electrodes disposed on the expandable section, wherein the expandable section is adapted to expand against an inner surface of a heart so as to position at least one of the plurality of electrodes at or near an SA node of the heart.

2. The lead of claim 1, wherein the expandable section includes a stent-like structure mounted over a balloon.

3. The lead of claim 2, wherein the expandable section includes a bell-shape.

4. The lead of claim 2, wherein the expandable section includes a funnel-shape.

5. The lead of claim 1, wherein the expandable section includes an expandable basket.

6. The lead of claim 1, wherein the expandable section is adapted to secure the electrodes proximate a junction of a superior vena cava and a right atrium.

7. The lead of claim 1, wherein the electrodes are adapted for mapping and pacing at the SA node.

8. The lead of claim 1, wherein the expandable section includes a preformed section of the lead, the preformed section having a shape adapted to secure the electrodes proximate a junction of a superior vena cava and a right atrium.

9. The lead of claim 8, wherein the preformed section includes a spiral shape.

10. The lead of claim 1, wherein the lead further includes a right ventricle electrode.

11. A lead comprising:
a lead body extending from a proximal end to a distal end;
an expandable section disposed proximate the distal end of the lead; and
a plurality of electrodes disposed on the expandable section, wherein the expandable section includes an expanded outer surface dimensioned to position at least one of the plurality of electrodes securely against or near an SA node.

12. The lead of claim 11, wherein each of the plurality of electrodes are independently controlled.

13. The lead of claim 11, wherein the expandable section includes a stent-like structure mounted over a balloon.

14. The lead of claim 11, wherein the expandable section includes an expandable basket.

15. The lead of claim 11, wherein the electrodes are adapted for mapping and pacing at the SA node.

16. The lead of claim 1, wherein the expandable section includes a preformed section of the lead.

17. The lead of claim 16, wherein the preformed section includes a spiral shape.

18. A method comprising:
positioning a plurality of electrodes within a heart near a junction of a superior vena cava and a right atrium;
mapping the heart using the plurality of electrodes; and
selectively choosing at least one of the electrodes to deliver energy pulses directly to an SA node or SA node conductive fibers.

19. The method of claim 18, wherein positioning includes expanding an expandable member on a lead, wherein the plurality of electrodes are exposed on a surface of the expandable section.

20. The method of claim 18, wherein mapping includes independently testing each of the plurality of electrodes to determine which of the electrodes is closest to the SA node.

21. The method of claim 18, wherein delivering energy pulses includes delivering pacing pulses.

22. A method comprising:
deploying an expandable member on a lead at a junction between a superior vena cava and a right atrium;
biasing a plurality of electrodes on the expandable section towards an SA node;
pacing the SA node using at least one of the plurality of electrodes.

23. The method of claim 22, wherein biasing includes inflating a balloon to expand a stent-like structure on the lead.

24. The method of claim 22, wherein biasing includes expanding a basket structure coupled to the lead.

25. The method of claim 22, wherein biasing includes allowing a pre-formed section of the lead to expand to its unbiased shape.
